(19) United States
(12) Patent Application Publication
(10) Pub. No.: US 2022/0401286 A1
(43) Pub. Date: Dec. 22, 2022

(54) WALKING TRAINING SYSTEM, CONTROL METHOD, AND PROGRAM

(71) Applicant: TOYOTA JIDOSHA KABUSHIKI KAISHA, Toyota-shi (JP)

(72) Inventors: Hiroshi SHIMADA, Tajimi-shi (JP); Taiga MATSUMOTO, Nagoya-shi (JP)

(73) Assignee: TOYOTA JIDOSHA KABUSHIKI KAISHA, Toyota-shi (JP)

(21) Appl. No.: 17/708,694

(22) Filed: Mar. 30, 2022

(30) Foreign Application Priority Data
Jun. 16, 2021 (JP) 2021-100037

(51) Int. Cl. A61H 3/00

(52) U.S. Cl. CPC A61H 3/008; A61H 2201/165; A61H 2201/5061; A61H 2201/5069; A61H 2201/5092; A61H 2201/1642

(57) ABSTRACT

A walking training system includes: a tension unit that pulls a leg of a trainee upward and forward; a sensor provided for determining a start timing of a swinging end phase of the leg; and a control unit that reduces a tensile force of the tension unit from the start timing of the swinging end phase.

### CROSS-REFERENCE TO RELATED APPLICATION

This application claims priority to Japanese Patent Application No. 2021-100037 filed on Jun. 16, 2021, incorporated herein by reference in its entirety.

### BACKGROUND

### 1. Technical Field

The present disclosure relates to a walking training system, a control method, and a program.

### 2. Description of Related Art

A walking training device is disclosed (see, for example, Japanese Unexamined Patent Application Publication No. 2017-35220 (JP 2017-35220 A) and Japanese Unexamined Patent Application Publication No. 2018-75301 (JP 2018-75301 A)). In JP 2017-35220 A and JP 2018-75301 A, a tension unit including a wire and a motor provides a tensile force to a leg of the user. In JP 2017-35220 A, the tension unit generates an additional tensile force at the start of forward swing of the leg or during the period of forward swing. In JP 2018-75301 A, the control device calculates the inertial force from the acceleration and the weight at the center-of-gravity position of the walking assist device. The control device controls the front and rear tension units so as to reduce the inertial force.

### SUMMARY

In such a walking training device, walking training can be performed more effectively by applying a more appropriate tensile force. For example, if a tensile force is applied at a timing when assist is unnecessary, it becomes difficult to perform effective training.

The present disclosure has been made to solve such a problem, and provides a walking training system, a control method, and a program for appropriately performing walking training.

A walking training system according to the present embodiment includes: a tension unit that pulls a leg of a trainee upward and forward; a sensor provided for determining a start timing of a swinging end phase of the leg; and a control unit that reduces a tensile force of the tension unit from the start timing of the swinging end phase.

In the above walking training system, a timing at which a lower leg of the leg has become perpendicular to a horizontal plane in a side view may be determined as the start timing of the swinging end phase.

In the above walking training system, in a period from the start timing of the swinging end phase to a timing of becoming standing, the tensile force with respect to the leg may be gradually reduced.

In the above walking training system, the sensor may be attached to a leg brace attached to the leg for detecting a knee joint angle of the leg.

In the above walking training system, the sensor may include a camera disposed to take an image of the leg from a side of the trainee.

A method for controlling a walking training system according to the present embodiment includes: a step of pulling a leg of a trainee upward and forward with a tension unit; a step of determining a start timing of a swinging end phase of the leg based on a detection result of a sensor; and a step of reducing a tensile force of the tension unit from the start timing of the swinging end phase.

In the above method, a timing at which a lower leg of the leg has become perpendicular to a horizontal plane in a side view may be determined as the start timing of the swinging end phase.

In the above method, in a period from the start timing of the swinging end phase to a timing of becoming standing, the tensile force with respect to the leg may be gradually reduced.

In the above method, the sensor may be attached to a leg brace attached to the leg for detecting a knee joint angle of the leg, and the start timing of the swinging end phase may be determined based on the knee joint angle.

In the above method, the sensor may include a camera disposed to take an image of the leg from a side of the trainee, and the start timing of the swinging end phase may be determined based on the image taken by the camera.

A program according to the present embodiment causes a control computer of the walking training system to execute the method.

The present disclosure provides a walking training system, a control method, and a program for appropriately performing walking training.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a walking training system 1 according to the present embodiment;

FIG. 2 is a perspective view showing a configuration of a leg brace;

FIG. 3 is a control block diagram of the walking training system 1;

FIG. 4 is a diagram illustrating phases in a walking cycle;

FIG. 5 is a graph showing changes in tensile force in one walking cycle;

FIG. 6 is a diagram illustrating a specific example of a sensor for determination; and

FIG. 7 is a flowchart showing a method for controlling a walking training system.

### DETAILED DESCRIPTION OF EMBODIMENTS

Hereinafter, the present disclosure will be described through embodiments of the disclosure, but the disclosure according to the scope of the claims is not limited to the following embodiments. Moreover, not all of the configurations described in the embodiments are indispensable as means for solving the problem.

### System Configuration

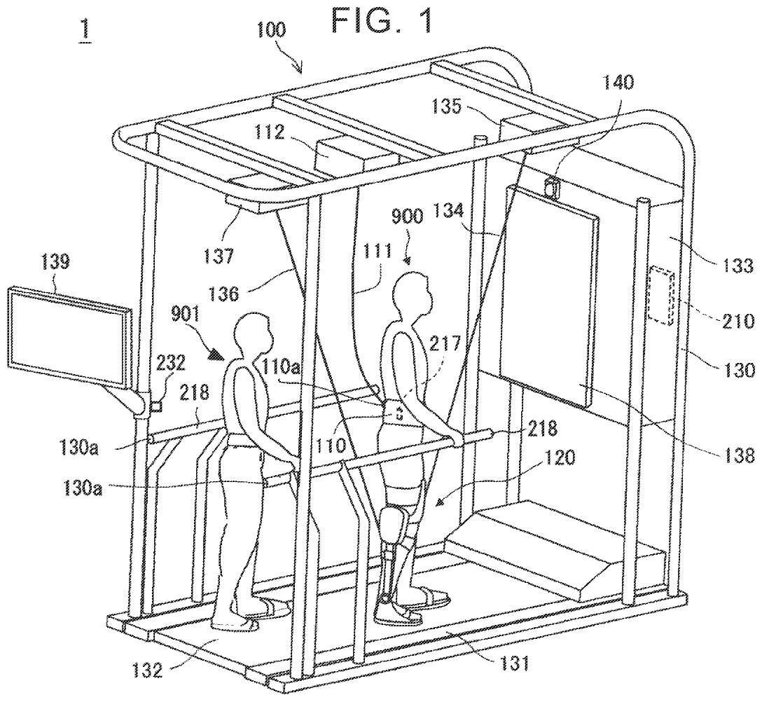

FIG. 1 is an overall conceptual diagram showing a configuration example of a rehabilitation (rehab) support system according to an embodiment. The rehab support system (walking training system 1) according to the present embodiment is mainly composed of a walking training device 100 and a leg brace 120.

The walking training device 100 is a specific example of a rehab support device that supports the rehab of a trainee (user) 900. The walking training device 100 is a device for the trainee 900 who is a hemiplegic patient suffering from paralysis in one leg to perform walking training in accordance with the guidance of a training staff 901. Here, the training staff 901 can be a therapist (physiotherapist) or a doctor, and assists the training of the trainee by guidance or caregiving. Therefore, the training staff 901 may be called a training instructor, a training caregiver, or a training assistant.

The walking training device 100 mainly includes a control panel 133 attached to a frame 130 constituting the entire skeleton, and a treadmill 131 on which the trainee 900 walks. The leg brace 120 is attached to an affected leg that is the leg of the trainee 900 on the paralyzed side. In FIG. 1, the leg brace 120 is attached to the right leg of the trainee 900.

The frame 130 is provided to stand on the treadmill 131 installed on the floor. The treadmill 131 rotates a ring-shaped belt 132 with a motor (not shown). The treadmill 131 is a device that prompts the trainee 900 to walk, and the trainee 900 who performs walking training rides on the belt 132 and attempts a walking motion in accordance with the movement of the belt 132. The training staff 901 can stand on the belt 132 behind the trainee 900 and perform a walking motion together with the trainee 900 as shown in FIG. 1, for example. However, it is usually preferable that the training staff 901 be in a state in which it is easy to perform caregiving to the trainee 900, that is, standing astride the belt 132.

The frame 130 supports a control panel 133 and a training monitor 138. The control panel 133 accommodates an overall control unit 210 that controls motors and sensors. The training monitor 138 is, for example, a liquid crystal panel, and presents the progress of training and the like to the trainee 900. Further, the frame 130 supports a front tension unit 135 at the front of the overhead portion of the trainee 900, a harness tension unit 112 at the overhead portion, and a rear tension unit 137 at the rear of the overhead portion. The frame 130 also includes handrails 130a for the trainee 900 to grab.

The handrails 130a are arranged on right and left sides of the trainee 900. Each handrail 130a is disposed to extend in a direction parallel to the walking direction of the trainee 900. The position of the handrail 130a in the up-down direction and the right-left direction can be adjusted. That is, the handrails 130a can include a mechanism for changing their height and width. Further, the handrail 130a can be configured such that the height of the handrail 130a is adjusted to make the height of the front side and the height of the rear side in the walking direction different so as to change the inclination angle thereof, for example. For example, the handrail 130a can be provided with an inclination angle that gradually increases along the walking direction.

Further, the handrail 130a is provided with a handrail sensor 218 for detecting the load received from the trainee 900. For example, the handrail sensor 218 can be a resistance change detection-type load detection sheet in which electrodes are arranged in a matrix. Further, the handrail sensor 218 can be a six-axis sensor in which a three-axis acceleration sensor (x, y, z) and a three-axis gyro sensor (roll, pitch, yaw) are combined. However, the type and the installation position of the handrail sensor 218 are not limited.

A camera 140 functions as an imaging unit for observing the whole body of the trainee 900. The camera 140 is installed near the training monitor 138 so as to face the trainee. The camera 140 captures still images and moving images of the trainee 900 during training. The camera 140 includes a set of a lens and an imaging element that provides such an angle of view that the whole body of the trainee 900 can be captured. The imaging element is, for example, a complementary metal-oxide-semiconductor (CMOS) image sensor that converts an optical image on an image plane into an image signal.

With the coordinated operation of the front tension unit 135 and the rear tension unit 137, the load of the leg brace 120 is offset so as not to be a burden on the affected leg, and further, the forward swing motion of the affected leg is assisted in accordance with the degree of the setting.

One end of a front wire 134 is connected to a winding mechanism of the front tension unit 135, and the other end is connected to the leg brace 120. The winding mechanism of the front tension unit 135 winds and unwinds the front wire 134 in accordance with the movement of the affected leg by turning on and off a motor (not shown). Similarly, one end of a rear wire 136 is connected to a winding mechanism of the rear tension unit 137, and the other end is connected to the leg brace 120. The winding mechanism of the rear tension unit 137 winds and unwinds the rear wire 136 in accordance with the movement of the affected leg by turning on and off a motor (not shown). With the coordinated operation of the front tension unit 135 and the rear tension unit 137, the load of the leg brace 120 is offset so as not to be a burden on the affected leg, and further, the forward swing motion of the affected leg is assisted in accordance with the degree of the setting.

The front wire 134 and the front tension unit 135 constitute a first tension unit that pulls the leg of the trainee 900 upward and forward. The rear wire 136 and the rear tension unit 137 constitute a second tension unit that pulls the leg of the trainee 900 upward and rearward. The front tension unit 135 and the rear tension unit 137 pull the front wire 134 and the rear wire 136, respectively, with a tensile force in accordance with the walking phase as described later. Further, the operation pattern of the tensile force may be set in accordance with the walking phase.

For example, as an operator, the training staff 901 sets the level of assistance to high, for a trainee who has severe paralysis. When the assist level is set to high, the front tension unit 135 winds up the front wire 134 with a relatively large force in accordance with the forward swing timing of the affected leg. As the training progresses and assistance becomes no longer needed, the training staff 901 sets the assist level to the minimum. When the assist level is set to the minimum, the front tension unit 135 winds up the front wire 134 with a force to cancel the weight of the leg brace 120 in accordance with the forward swing timing of the affected leg.

The walking training device 100 includes a fall prevention harness device serving as a safety device and including a brace 110, a harness wire 111, and a harness tension unit 112 as its main components. The brace 110 is a belt wrapped around the abdomen of the trainee 900 and is fixed to the waist portion by, for example, a hook-and-loop fastener. The brace 110 includes a connecting hook 110a for connecting one end of the harness wire 111 that is a hanger, and can also be referred to as a hanger belt. The trainee 900 wears the brace 110 such that the connecting hook 110a is located on the rear back portion.

One end of the harness wire 111 is connected to the connecting hook 110a of the brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 is about to fall, the fall prevention harness device winds up the harness wire 111 in accordance with the instruction of the overall control unit 210 that detects the movement, supports the upper body of the trainee 900 with the brace 110, and suppresses the trainee 900 from falling.

The brace 110 includes a posture sensor 217 for detecting the posture of the trainee 900. The posture sensor 217 is, for example, a combination of a gyro sensor and an acceleration sensor, and outputs an inclination angle of the abdomen on which the brace 110 is attached with respect to the direction of gravity.

The management monitor 139 is attached to the frame 130 and is a display input device mainly for monitoring and operation by the training staff 901. The management monitor 139 is, for example, a liquid crystal panel, and a touch panel is provided on the surface thereof. The management monitor 139 displays various menu items related to training settings, various parameter values at the time of training, training results, and the like. Further, an emergency stop button 232 is provided near the management monitor 139. When the training staff 901 presses the emergency stop button 232, an emergency stop of the walking training device 100 is performed.

The overall control unit 210 generates rehab data that can include setting parameters related to the training settings, various data related to leg movements output from the leg brace 120 as the training results, and the like. The rehab data can include data indicating the training staff 901 or his/her years of experience, skill level, and the like, data indicating the symptoms, walking ability, recovery level, and the like of the trainee 900, and various data output from sensors and the like provided outside the leg brace 120.

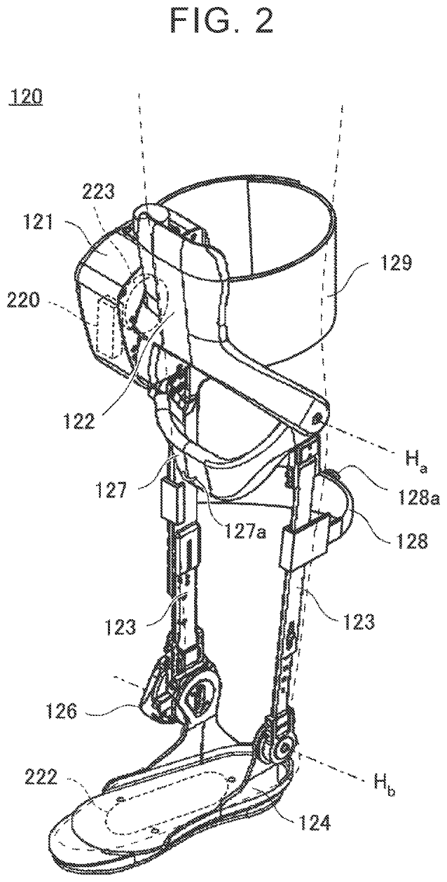

Next, the leg brace 120 will be described with reference to FIG. 2. FIG. 2 is a schematic perspective view showing a configuration example of the leg brace 120. The leg brace 120 mainly includes a control unit 121, a plurality of frames that support various parts of the affected leg, and a load sensor 222 for detecting a load applied to the sole.

The control unit 121 includes an auxiliary control unit 220 that controls the leg brace 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open and close around the hinge axis Hₐ. An angle sensor 223 accommodated in the control unit 121 is, for example, a rotary encoder, and detects the angle between the upper leg frame 122 and the lower leg frames 123 around the hinge axis Hₐ. The lower leg frames 123 and the foot flat frame 124 pivot relative to each other around a hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, around the center in the right-left direction. The rear connecting frame 128 is provided so as to extend in the right-left direction on the rear side of the lower leg and connect to the lower leg frames 123 at both ends. Further, the rear connecting frame 128 is provided with a connecting hook 128a for connecting the rear wire 136, around the center in the right-left direction.

The upper leg frame 122 is provided with an upper leg belt 129. The upper leg belt 129 is a belt integrally provided on the upper leg frame, and is wrapped around the upper leg portion of the affected leg to fix the upper leg frame 122 to the upper leg portion. This suppresses the entire leg brace 120 from shifting with respect to the leg of the trainee 900.

The load sensor 222 is a load sensor embedded in the foot flat frame 124. The load sensor 222 can also be configured to detect the magnitude and the distribution of the vertical load received by the sole of the trainee 900 to detect a center of pressure (COP), for example. The load sensor 222 is a resistance change detection-type load detection sheet in which the electrodes are arranged in a matrix, for example.

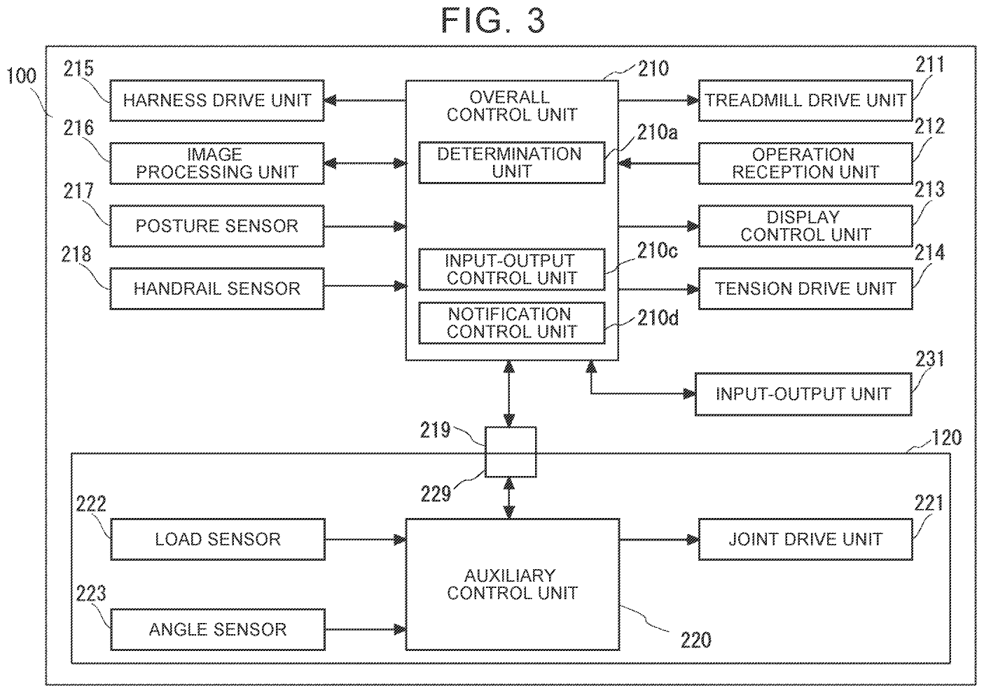

Next, a system configuration example of the walking training device 100 will be described with reference to FIG. 3. FIG. 3 is a block diagram showing the system configuration example of the walking training device 100. As shown in FIG. 3, the walking training device 100 can include an overall control unit 210, a treadmill drive unit 211, an operation reception unit 212, a display control unit 213, and a tension drive unit 214. The walking training device 100 can also include a harness drive unit 215, an image processing unit 216, the posture sensor 217, the handrail sensor 218, a communication connection interface (IF) 219, an input-output unit 231, and the leg brace 120.

The overall control unit 210 is, for example, a micro processing unit (MPU), and executes control of the entire device by executing a control program read from a system memory. The overall control unit 210 can include a determination unit 210a, an input-output control unit 210c, and a notification control unit 210d, which will be described later.

The treadmill drive unit 211 includes a motor for rotating the belt 132 and a drive circuit thereof. The overall control unit 210 executes rotation control of the belt 132 by transmitting a drive signal to the treadmill drive unit 211. The overall control unit 210 adjusts the rotation speed of the belt 132 in accordance with, for example, the walking speed set by the training staff 901.

The operation reception unit 212 receives an input operation from the training staff 901 and transmits an operation signal to the overall control unit 210. The training staff 901 operates operation buttons provided on the device, a touch panel superimposed on the management monitor 139, the attached remote controller, and the like that constitute the operation reception unit 212. Through this operation, it is possible to give an instruction to turn on and off the power supply and start training, input a numerical value related to the setting, and select a menu item. The operation reception unit 212 can also receive an input operation from the trainee 900.

The display control unit 213 receives a display signal from the overall control unit 210, generates a display image, and displays the image on the training monitor 138 or the management monitor 139. The display control unit 213 generates an image showing the progress of training and a real-time image captured by the camera 140 in accordance with the display signal.

The tension drive unit 214 includes a motor for pulling the front wire 134 and a drive circuit thereof that constitute the front tension unit 135, and a motor for pulling the rear wire 136 and a drive circuit thereof that constitute the rear tension unit 137. The overall control unit 210 controls the winding of the front wire 134 and the winding of the rear wire 136 by transmitting a drive signal to the tension drive unit 214. Further, the overall control unit 210 controls the tensile force of each wire by controlling the driving torque of the motor, as well as the winding operation. The overall control unit 210 detects the timing of the affected leg in the walking cycle based on the detection results of the load sensor 222 and the angle sensor, and increases or decreases the tensile force of each wire in synchronization with that timing, thereby assisting the forward swing motion of the affected leg.

The harness drive unit 215 includes a motor for pulling the harness wire 111 and a drive circuit thereof that constitute the harness tension unit 112. The overall control unit 210 controls the winding of the harness wire 111 and the tensile force of the harness wire 111 by transmitting a drive signal to the harness drive unit 215. For example, when the trainee 900 is predicted to fall, the overall control unit 210 winds up the harness wire 111 by a certain amount to suppress the trainee from falling.

The image processing unit 216 is connected to the camera 140 and can receive an image signal from the camera 140. The image processing unit 216 receives an image signal from the camera 140 and performs image processing on the received image signal to generate image data, in accordance with the instruction from the overall control unit 210. Further, the image processing unit 216 can also perform image processing on the image signal received from the camera 140 to execute a specific image analysis, in accordance with the instruction from the overall control unit 210. For example, the image processing unit 216 detects the position of the foot (standing position) of the affected leg that is in contact with the treadmill 131 by image analysis. Specifically, for example, the standing position is calculated by extracting an image region around the tip of the foot flat frame 124 and analyzing an identification marker drawn on the belt 132 and overlapping the tip portion.

As described above, the posture sensor 217 detects the inclination angle of the abdomen of the trainee 900 with respect to the direction of gravity, and transmits the detection signal to the overall control unit 210. The overall control unit 210 calculates the posture of the trainee 900, specifically the inclination angle of the trunk, using the detection signal from the posture sensor 217. The overall control unit 210 and the posture sensor 217 may be connected by wire or by short-range wireless communication.

The handrail sensor 218 detects a load applied to the handrail 130a. That is, a load corresponding to a portion of the weight of the trainee 900 that the trainee 900 cannot support with both legs is applied to the handrail 130a. The handrail sensor 218 detects this load and transmits a detection signal to the overall control unit 210.

The overall control unit 210 also plays a role as a function execution unit that executes various calculations related to the control and performs the control. The determination unit 210a determines the walking phase in the walking cycle using the data acquired from various sensors.

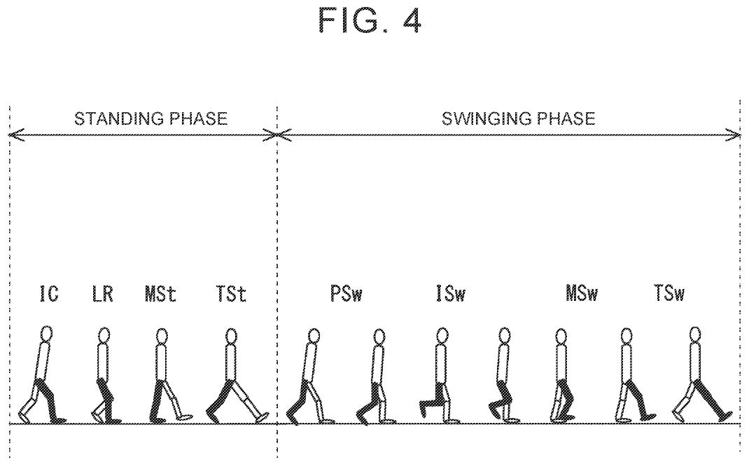

The phases in the walking cycle will be described with reference to FIG. 4. FIG. 4 is a side view schematically showing the phases in the walking cycle. Here, the phases of walking are based on the Rancho Los Amigos method. Further, the swinging phase and the standing phase are defined with the right leg to which the leg brace 120 is attached as a reference. That is, a period when the right leg is off the floor is defined as the swinging phase, and a period when the right leg is in contact with the floor is defined as the standing phase. It is assumed that the floor surface is a horizontal plane.

In the order from the start, the standing phase includes an initial contact IC (initial ground contact), a loading response LR (load response phase), a mid-stance MSt (standing middle phase), and a terminal stance TSt (standing end phase). In the order from the start, the swinging phase includes a pre-swing PSw (pre-swinging phase), an initial swing ISw (swinging initial phase), a mid-swing MSw (swinging middle phase), and a terminal swing TSw (swinging end phase). Thus, one walking cycle is divided into eight phases.

The initial contact IC is the moment when the right foot contacts the ground, and serves as the end and the start of the walking cycle. The loading response LR is a period from the initial contact IC to the moment when the left foot has left the ground. The mid-stance MSt is a period from the moment when the left foot has left the ground to the moment when the heel of the right foot has left the floor. The terminal stance TSt is a period from the moment when the heel of the right foot has left the ground to the initial contact IC of the left leg (the moment when the left foot contacts the ground).

The pre-swing PSw is a period from the initial contact IC of the left leg (the moment when the left foot contacts the ground) to the moment when the toe of the right foot has left the floor. The initial swing ISw is a period from the moment when the toe of the right foot has left the floor to the moment when the leg joints on both sides cross in the sagittal plane. The mid-swing MSw is a period from the moment when the leg joints on both sides cross in the sagittal plane to the moment when the lower leg of the right leg has become perpendicular to the floor. The terminal swing TSw is a period from the moment when the lower leg of the right leg has become perpendicular to the floor to the initial contact IC of the right leg (the moment when the right foot contacts the ground). In this way, one walking cycle is a period of two steps in total including one step on each side.

The communication connection IF 219 is an interface connected to the overall control unit 210, and is an interface for providing a command to the leg brace 120 attached to the affected leg of the trainee 900 and receiving sensor information.

The leg brace 120 can include a communication connection IF 229 that is connected to the communication connection IF 219 by wire or wirelessly. The communication connection IF 229 is connected to the auxiliary control unit 220 of the leg brace 120. The communication connection IF 219 and the communication connection IF 229 are communication interfaces such as a wired local area network (LAN) or a wireless LAN conforming to the communication standards.

The leg brace 120 can include the auxiliary control unit 220, the joint drive unit 221, the load sensor 222, and the angle sensor 223. The auxiliary control unit 220 is, for example, an MPU, and controls the leg brace 120 by executing the control program provided by the overall control unit 210. Further, the auxiliary control unit 220 notifies the overall control unit 210 of the state of the leg brace 120 via the communication connection IF 219 and the communication connection IF 229. Further, the auxiliary control unit 220 receives a command from the overall control unit 210 and executes control of starting, stopping, and the like of the leg brace 120.

The joint drive unit 221 includes a motor of the control unit 121 and a drive circuit thereof. The auxiliary control unit 220 transmits the drive signal to the joint drive unit 221 to force the upper leg frame 122 and the lower leg frames 123 to relatively open or close around the hinge axis Hₐ. Such motions assist the extension motion and the bending motion of the knee and suppress knee collapse.

As described above, the load sensor 222 detects the magnitude and the distribution of the vertical load received by the sole of the trainee 900, and transmits the detection signal to the auxiliary control unit 220. The auxiliary control unit 220 receives and analyzes the detection signal to determine the state of the swinging and standing and estimate the switching.

As described above, the angle sensor 223 detects the angle between the upper leg frame 122 and the lower leg frames 123 around the hinge axis Hₐ, and transmits the detection signal to the auxiliary control unit 220. The auxiliary control unit 220 receives this detection signal and calculates the opening angle of the knee joint.

The input-output unit 231 includes, for example, a universal serial bus (USB) interface, and is a communication interface for connecting to external devices (an external communication device 300 and other external devices). The input-output control unit 210c of the overall control unit 210 communicates with the external devices via the input-output unit 231, rewrites the control program in the overall control unit 210 and the control program in the auxiliary control unit 220 described above, receives commands, and outputs generated rehab data, for example. The walking training device 100 communicates with a server 500 via the input-output unit 231 and the external communication device 300 under the control of the input-output control unit 210c. For example, the input-output control unit 210c can execute control of transmitting the rehab data to the server 500 and control of receiving commands from the server 500, via the input-output unit 231 and the external communication device 300.

The notification control unit 210d performs notification from the management monitor 139 or a separately provided speaker by controlling the display control unit 213, a separately provided audio control unit, or the like, at the scene where notification to the training staff 901 becomes necessary. Details of the notification will be described later, but the scene where the notification to the training staff 901 becomes necessary may be a case where a command for performing the notification is received from the server 500.

The determination unit 210a determines each phase in the walking cycle based on the detection results of various sensors. For example, the determination unit 210a determines the start timing of the terminal swing TSw (swinging end phase) of the right leg. Then, the determination unit 210a outputs the determination result to the tension drive unit 214. For example, the timing at which the lower leg of the right leg becomes perpendicular to the horizontal plane in the side view is defined as the start timing of the swinging end phase.

As described above, the determination unit 210a collects the detection data of various sensors. The determination unit 210a determines the walking phase by analyzing changes over time of the detection data of the sensors. As the sensors for determining the phase in the walking cycle, for example, the angle sensor 223 and the load sensor 222 are used. Of course, the determination unit 210a may make a determination based on the detection data of one sensor, or may make a determination based on the detection data of a plurality of sensors.

The tension drive unit 214 outputs a tensile force command value for driving the wire to a motor and the like based on the determination result of the determination unit 210a. Thereby, the wire can apply an appropriate tensile force in accordance with the walking phase. Accordingly, the walking motion can be assisted with an appropriate tensile force in accordance with the walking phase.

For example, the overall control unit 210 controls the tension drive unit 214 such that the tensile force of the front wire 134 is reduced from the start timing of the terminal swing TSw (swinging end phase). Thereby, the tensile force on the forward swing side can be reduced at the swinging end phase. Thus, it is possible to suppress the leg from being unnecessarily swung forward or lifted at the swinging end phase. This allows natural transition to the standing phase. Since the tension drive unit 214 can provide an appropriate tensile force in the swinging end phase in which assist is unnecessary, the trainee 900 can perform effective training.

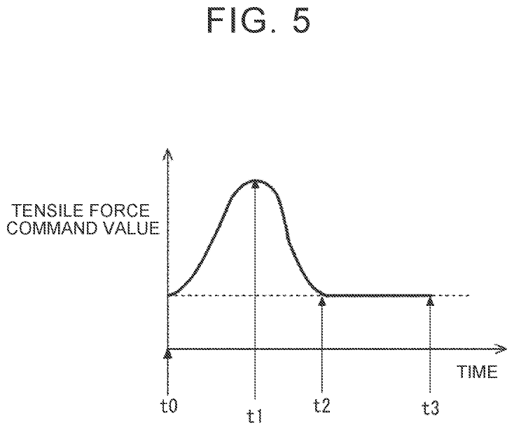

FIG. 5 is a diagram showing an example of a pattern of the tensile force command values provided to the front tension unit 135. In FIG. 5, time t0 is the start timing of forward swing, that is, the start timing of the swinging phase. Specifically, time t0 corresponds to the moment when the toe of the right foot has left the floor. Further, time t1 in FIG. 5 is the start timing of the swinging end phase. Specifically, time t1 corresponds to the timing when the lower leg of the right leg has become perpendicular to the horizontal plane. Time t2 in FIG. 5 is the timing of becoming the standing phase, that is, the initial contact IC. Specifically, time t2 corresponds to the timing when the right foot contacts the ground. Time t3 corresponds to time t0 of the next walking cycle. Time t0 to t2 corresponds to the swinging phase, and t2 to t3 corresponds to the standing phase.

In FIG. 5, the tension drive unit 214 outputs the tensile force command value such that the tensile force gradually decreases in the period from time t1 to time t2. The overall control unit 210 controls the tension drive unit 214 such that the tensile force decreases, triggered by the start timing of the swinging end phase. From time t1 to time t2, the tensile force of the front wire 134 monotonically decreases.

Then, the tensile force command value is constant from time t2 to time t3. That is, the tensile force of the front wire 134 is constant during the standing phase. The tensile force is constant from the initial contact IC of the right leg to the initial contact IC of the left leg. The tensile force in the standing phase is equal to or less than the tensile force in the swinging phase. The tension drive unit 214 outputs the tensile force command value such that the tensile force gradually increases in the period from time t0 to time t1. Thus, the tensile force of the front wire 134 monotonically increases from time t0 to time t1. The tensile force increases from the pre-swing PSw to the mid-swing MSw. The tensile force becomes maximum at time t1.

In this way, the determination unit 210a determines the walking phase in the walking cycle based on the detection results of the sensors. The overall control unit 210 controls the tensile force of the tension drive unit 214 from the determination result of the walking phase by the determination unit 210a. Thereby, it is possible to suppress unnecessary tensile force from being applied in the swinging end phase. Further, since the walking phase is determined from the detection results of the sensors, the walking phase can be detected accurately. Thus, the tension drive unit 214 can apply an appropriate tensile force in accordance with the actual gait.

The determination unit 210a does not have to detect all of the initial contact IC, the loading response LR, the mid-stance MSt, the terminal stance TSt, the pre-swing PSw, the initial swing ISw, the mid-swing MSw, and the terminal swing TSw. That is, the determination unit 210a only needs to detect at least one of the initial contact IC, the loading response LR, the mid-stance MSt, the terminal stance TSt, the pre-swing PSw, the initial swing ISw, the mid-swing MSw, and the terminal swing TSw. For the definitions of the walking phases in the walking cycle, a method other than the Rancho Los Amigos method may be used.

Further, in the present embodiment, the angle sensor 223 for detecting the knee joint angle is attached to the leg brace 120. Therefore, the angle sensor 223 can accurately detect the knee joint angle. Based on the detection result of the angle sensor 223, the determination unit 210a determines the start timing of the swinging end phase. This makes it possible to improve the determination accuracy of the walking phase. Therefore, the tension drive unit 214 can apply an appropriate tensile force forward and upward. Further, the determination unit 210a determines the timing at which the lower leg of the leg becomes perpendicular to the horizontal plane, as the start timing of the swinging end phase. Thereby, the walking phase can be appropriately determined.

As described above, the angle sensor 223 can be used as the sensor provided for determining the start timing of the swinging end phase of the leg (hereinafter referred to as a sensor for determining the walking phase). Of course, the sensor for determining the walking phase is not limited to the angle sensor 223. For example, the load sensor 222 shown in FIGS. 2 and 3 may be used as the sensor for determining the walking phase.

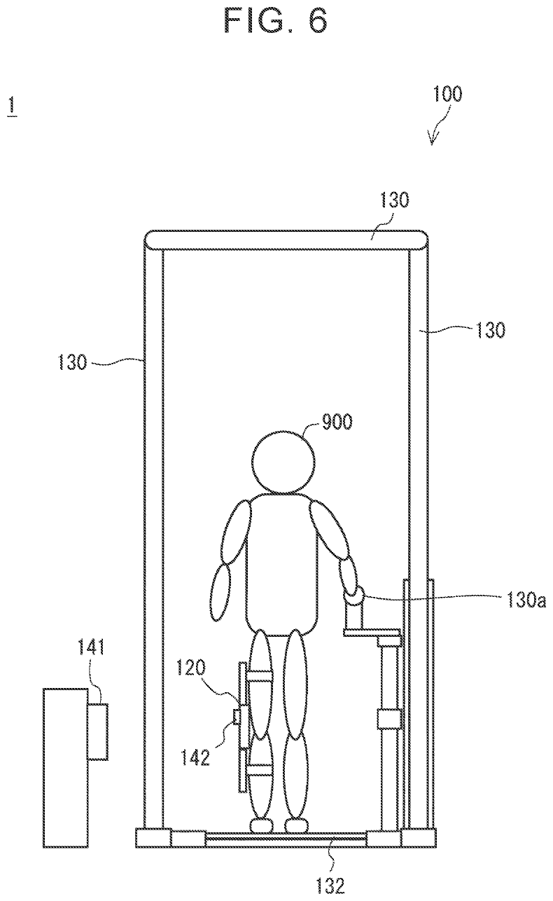

Further, as shown in FIG. 6, the gyro sensor 142 provided on the leg brace 120 may be used as the sensor for determining the walking phase. FIG. 6 is a front view schematically showing an example of sensor arrangement. In FIG. 6, the configurations such as the front wire 134, the front tension unit 135, and the like of the walking training system 1 are appropriately omitted.

The gyro sensor 142 detects the angular velocity of the leg. By using the sensor attached to the leg brace 120 as the sensor for determining the walking phase, the determination accuracy can be improved. That is, the walking phase can be accurately determined. Therefore, the tension drive unit 214 can apply a more appropriate tensile force in accordance with the walking cycle. Further, the leg brace 120 may be provided with an acceleration sensor that detects the acceleration of the leg.

Alternatively, the camera 141 that takes an image of the legs of the user may be used as the sensor for determining the walking phase. For example, the camera 141 is disposed so as to take an image of the legs from the side of the trainee 900. The camera 141 is disposed outside the frame 130 so as not to interfere with the training. The determination unit 210a determines the walking phase based on the image taken by the camera 141. This can improve the determination accuracy. The determination unit 210a may make a determination using only the image taken by the camera 141, or may make a determination based on the image taken by the camera 141 and the detection result of another sensor. Further, another motion sensor may be used as the sensor for determining the walking phase.

Further, two or more sensors may be combined and used as the sensor for determining the walking phase. For example, the determination unit 210a may determine the start timing of the swinging end phase of the leg based on the detection result of the gyro sensor 142 and the detection result of the angle sensor 223. The determination unit 210a detects the timing at which the lower leg of the leg becomes perpendicular to the horizontal plane based on the detection results of the two sensors. Then, the detected timing is set as the start timing of the swinging end phase. Thereby, the walking phase can be simply and appropriately determined. Of course, the sensor for determining the walking phase may be a sensor other than the above. Further, a sensor other than the above may be combined with the above-mentioned sensors and used as the sensor for determining the walking phase.

Further, the determination unit 210a may estimate the start timing of the swinging end phase of the leg from the timing at which the knee joint has bent. For example, the determination unit 210a may determine the timing a predetermined time after the timing at which the knee joint has bent, as the start timing of the swinging end phase. Alternatively, the determination unit 210a may estimate the start timing of the swinging end phase from the swing speed of the leg. For example, the determination unit 210a calculates the swing speed of the leg based on the detection results of the gyro sensor 142 and the like. Alternatively, the determination unit 210a may obtain the start timing of the swinging end phase from the swing speed of the leg.

In the above description, the leg brace 120 is attached to the right leg, but it may be attached to the left leg. Moreover, the leg brace 120 may be attached to both legs. Furthermore, the leg brace 120 functions as the walking assist device including the joint drive unit 221 for driving the knee joint, the auxiliary control unit 220, and the like, but the configuration of the leg brace 120 is not particularly limited. For example, the leg brace 120 may include only a passive joint mechanism.

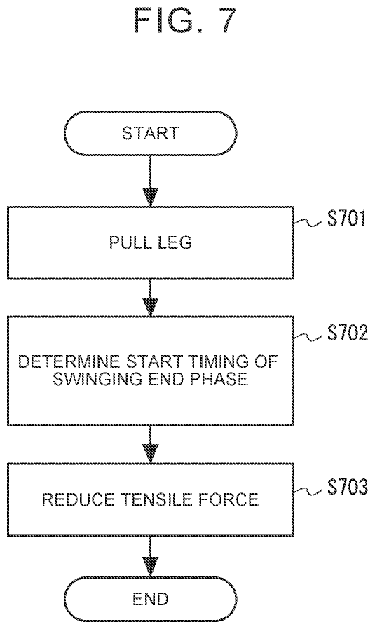

A method for controlling the walking training system 1 according to the present embodiment will be described with reference to FIG. 7. FIG. 7 is a flowchart showing the control method. First, the tension drive unit 214 pulls the leg forward and upward (S701). Then, the determination unit 210a determines the start timing of the swinging end phase based on the detection results of the sensors (S702). Then, the tension drive unit 214 reduces the tensile force (S703). The above processes may be repeated until the end of the walking training. That is, the control pattern of the tensile force command value becomes the same in each walking cycle.

By the control method according to the present embodiment, it is possible to suppress an excessive tensile force from being applied in the swinging end phase. Further, the walking phase is determined from the detection results of the sensors, so the walking phase can be detected accurately. Thus, the tension drive unit 214 can apply an appropriate tensile force in accordance with the actual gait.

The operation method of the above walking training system can be implemented by a computer program or hardware. The overall control unit 210 includes a memory for storing the program, a processor for executing the program, and the like. As the overall control unit 210 executes the program, the operation method of the walking training system 1 according to the present embodiment can be executed.

Part or all of the above processes may be executed by a computer program. That is, the control of the walking training system 1 is executed as the control computer constituting the overall control unit 210 executes the program. The above program includes instructions (or software codes) for causing the computer to perform one or more of the functions described in the embodiments when loaded into the computer. The program may be stored in a non-transitory computer-readable medium or a tangible storage medium. Examples of the computer-readable medium or the tangible storage medium include, but not limited to, a random-access memory (RAM), a read-only memory (ROM), a flash memory, a solid-stated drive (SSD) or other memory technologies, a compact disc read-only memory (CD-ROM), a digital versatile disc (DVD), a Blu-ray (registered trademark) disc, or other optical disc storages, a magnetic cassette, a magnetic tape, a magnetic disc storage, or other magnetic storage devices. The program may be transmitted on a transitory computer-readable medium or a communication medium. Examples of the transitory computer-readable medium or the communication medium include, but not limited to, electrical, optical, acoustic, or other forms of propagating signals.

Although the disclosure made by the present inventors has been specifically described based on the embodiments, it is needless to say that the present disclosure is not limited to the above embodiments and can be variously modified without departing from the scope thereof.

What is claimed is:

1. A walking training system comprising:
a tension unit that pulls a leg of a trainee upward and forward;
a sensor provided for determining a start timing of a swinging end phase of the leg; and
a control unit that reduces a tensile force of the tension unit from the start timing of the swinging end phase.

2. The walking training system according to claim 1, wherein a timing at which a lower leg of the leg has become perpendicular to a horizontal plane in a side view is determined as the start timing of the swinging end phase.

3. The walking training system according to claim 1, wherein in a period from the start timing of the swinging end phase to a timing of becoming standing, the tensile force with respect to the leg is gradually reduced.

4. The walking training system according to claim 1, wherein the sensor is attached to a leg brace attached to the leg for detecting a knee joint angle of the leg.

5. The walking training system according to claim 1, wherein the sensor includes a camera disposed to take an image of the leg from a side of the trainee.

6. A method for controlling a walking training system, the method comprising:
a step of pulling a leg of a trainee upward and forward with a tension unit;
a step of determining a start timing of a swinging end phase of the leg based on a detection result of a sensor; and
a step of reducing a tensile force of the tension unit from the start timing of the swinging end phase.

7. The method according to claim 6, wherein a timing at which a lower leg of the leg has become perpendicular to a horizontal plane in a side view is determined as the start timing of the swinging end phase.

8. The method according to claim 6, wherein in a period from the start timing of the swinging end phase to a timing of becoming standing, the tensile force with respect to the leg is gradually reduced.

9. The method according to claim 6, wherein:
the sensor is attached to a leg brace attached to the leg for detecting a knee joint angle of the leg; and
the start timing of the swinging end phase is determined based on the knee joint angle.

10. The method according to claim 6, wherein:
the sensor includes a camera disposed to take an image of the leg from a side of the trainee; and
the start timing of the swinging end phase is determined based on the image taken by the camera.

11. A program for causing a control computer of the walking training system to execute the method according to claim 6.
